(19) United States
(12) Patent Application Publication
(10) Pub. No.: US 2013/0052244 A1
(43) Pub. Date: Feb. 28, 2013

(54) Stem Cell Compositions and Methods

(71) Applicants: Mark Weinreb, Woodbury, NY (US); Francisco Javier Silva, Miramar, FL (US)

(72) Inventors: Mark Weinreb, Woodbury, NY (US); Francisco Javier Silva, Miramar, FL (US)

(21) Appl. No.: 13/599,514

(22) Filed: Aug. 30, 2012

Related U.S. Application Data
(60) Provisional application No. 61/529,195, filed on Aug. 30, 2011.

(51) Int. Cl. A61K 8/97; A61Q 19/08; A61K 8/14

(52) U.S. Cl. 424/401; 424/744; 424/725

(57) ABSTRACT

Provided are cosmetic compositions including a succulent plant stem cell extract and, optionally, an excipient. Also provided are methods of preparing a cosmetic composition. The methods include, for example, growing an Aloe vera callus to a predetermined size, preparing an extract from the callus, and mixing the extract with an excipient, thereby preparing a cosmetic composition.

### CROSS REFERENCE TO RELATED APPLICATIONS

This application claims priority to and the benefit of U.S. Provisional Application No. 61/529,195, filed on Aug. 30, 2011, the entire disclosure of which is incorporated by reference herein.

### BACKGROUND

Aloe vera is a species of succulent plant in the genus Aloe. Aloe medications can be taken by mouth or applied to the skin. Aloe gel can be taken by mouth for osteoarthritis, bowel diseases including ulcerative colitis, fever, itching and inflammation, and as a general tonic. It is also used for stomach ulcers, diabetes, asthma, and for treating some side effects of radiation treatment. Aloe gel can also be used topically, as a remedy for skin conditions including burns, sunburn, frostbite, psoriasis, and cold sores.

### SUMMARY OF THE INVENTION

Described are cosmetic compositions that include succulent plant stem cells, such as Aloe vera stem cells, or extracts thereof. The compositions are useful for cosmetic or therapeutic applications to treat a wide range of skin conditions, such as dermatological signs of aging. Also described are methods of making such cosmetic compositions.

In one aspect, the invention provides a cosmetic composition comprising about 25% to about 90% of a succulent plant stem cell extract by weight of the composition. In some embodiments, the plant extract is derived from at least one succulent family selected from the group consisting of: Aloaceae, Agavaceae, Cactaceae, Crassulaceae, Aizoaceae, Apocynaceae, Didiereaceae, Euphorbiaceae, Asphodelaceae, and Portulacaceae. In some embodiments, the composition further comprises a carrier selected from a group consisting of an emollient, an emulsion, a liposome, and a gel. In some embodiments, the composition is administered as a cosmetic, subcutaneously, intraperitoneally, topically, transdermally, transmucosally, intramuscularly, intranasally or intravenously.

In another aspect, the invention provides a cosmetic formulation comprising an extract from an Aloe vera callus weighing about 0.5 g to about 1 g, and an excipient.

In another aspect, the invention provides a method of preparing a cosmetic composition comprising the steps of growing an Aloe vera callus to a weight of about 0.5 g to about 1 g, preparing an extract from the callus, and mixing the extract with an excipient, thereby preparing a cosmetic composition. In some embodiments, the extract to excipient ratio is from about 5% to about 99% by weight. In some embodiments, the invention includes a cosmetic composition prepared by any of the foregoing methods.

### Stem Cell Compositions

Compositions described herein include stem cells from succulent plants or extracts thereof.

In general, stem cells useful in the methods described herein can be obtained from a succulent plant or succulent plant tissue. Stem cells can be derived from any succulent plant, including, e.g., plants of the genus Aloe, e.g., Aloe vera. Additional succulent plants include plants in the following families: Agavaceae, Cactaceae, Crassulaceae, Aizoaceae, Apocynaceae, Didiereaceae, Euphorbiaceae, Asphodelaceae, and Portulacaceae.

Stem cells or stem-cell containing plant tissue can be derived from any appropriate part of a succulent plant, including, e.g., meristematic tissue, apical meristems, shoot meristems, vegetative meristems, axillary shoots, decapitated shoot explants, immature inflorescence, shoot tips, nodal buds, or callus formations. For example, a callus from a succulent plant can be grown under appropriate culture conditions to a weight of about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, or more. Stem cells can be obtained from the callus, or an extract can be prepared from the callus. In some embodiments, an extract is prepared from more than one callus, e.g., 2, 3, 4, 5, 10, 15, 20, or more, calluses. Methods of culturing plant cells are known in the art (see, e.g., Roberta, Plant Tissue Culture: Techniques and Experiments, 2d ed., Acad. Press (2000) and Trigiano and Gray, eds., Plant Tissue Culture Concepts and Laboratory Exercises, 2d ed., CRC Press (2000)).

Extracts of plant tissue, e.g., stem cell-containing plant tissue (such as a callus) can be prepared using known methods (see, e.g., Liu et al., J. Biomed. Biotechnol. 2010: 479426 (2010)). In some instances, an extract is produced by solvent extraction. Solvents that can be used include, e.g., polar and non-polar organic solvents, water, and mixtures thereof. Particular solvents include acetone, water, ethanol and mixtures thereof. Solvent extracted components may be subject to further purification/separation steps such as chromatography or fractional distillation.

In some embodiments, an extract is prepared by homogenization of stem cell-containing plant tissue, e.g., a callus. The homogenate can be concentrated to a particular concentration using known methods. For example, an extract can be concentrated to a level of about 0.01 mg/L to about 50 mg/L.

In other embodiments, the extract is dried using known methods (e.g., lyophilizing, freeze-drying). The dried extract can then be reconstituted prior to inclusion in a composition described herein. For example, the dried extract can be reconstituted to a concentration of about 0.01 to about 50 mg/L.

In some instances, an extract described herein includes polysaccharides, mannans, anthraquinones, lectins, and/or plant-based exozomes.

Stem cells isolated from succulent plants, or extracts from stem cell-containing plant tissue (e.g., a callus) can be incorporated into compositions described herein. In some instances, a composition includes from about 5% to about 100% extract by weight. For example, the composition can include about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% extract by weight.

### Formulations

The compositions described herein may be administered systemically or locally, e.g., topically. For topical application, the compositions of the present disclosure can include a medium compatible with a stem cell, extract or subject. Such topical compositions can exist in many forms, e.g., in the form of a solution, gel, cream, ointment, paste, gel, lotion, shampoo, stick, soap or aerosol. In particular instances, a composition described herein is formulated as a cosmetic.

A wide variety of carrier materials can be employed in the compositions described herein. For example, topical compositions can include a pharmaceutically-acceptable aqueous or organic solvent. Examples of suitable organic solvents include: propylene glycol, polyethylene glycol (200-600), polypropylene glycol (425-2025), poly vinyl pyrrolidine, propylene glycol-14 butyl ether, glycerol, 1,2,4-butanetriol, sorbitol esters, 1,2,6-hexanetriol, ethanol, isopropanol, butanediol, and mixtures thereof. Other carrier materials include alcohols, allantoin, glycerin, vitamin A and E oils, mineral oils, and polyethylene glycols. Other additives, e.g., preservatives, fragrance, sunscreen, or other cosmetic ingredients, can be present in the composition. These product types may comprise several types of carrier systems including, but not limited to solutions, emulsions, gels and solids.

Topical compositions may be formulated as a solution comprising an emollient, i.e., a material used for the prevention or relief of dryness, as well as for the protection of the skin. A wide variety of suitable emollients are known and may be used herein (see Sagarin, Cosmetics, Science and Technology 2nd Edition, Vol. 1, pp. 32-43 (1972)). Such compositions can contain from about 2% to about 50% of a topical pharmaceutically-acceptable emollient.

A topical composition described herein can be formulated as an emulsion in which from about 1% to about 10%, or from about 2% to about 5%, of the carrier system comprises an emulsifier. Emulsifiers may be non-ionic, anionic or cationic. Suitable emulsifiers are disclosed in, for example, McCutcheon's Detergents and Emulsifiers, North American Edition, pages 317-324 (1986). Single emulsion skin care preparations, such as lotions and creams, of the oil-in-water type and water-in-oil type are well known in the cosmetic art. Such emulsions can stabilize and enhance the penetration of actives. Multiphase emulsion compositions, such as the water-in-oil-in-water type may also be used. In general, such single or multiphase emulsions contain water, emollients and emulsifiers as essential ingredients.

Another emulsion carrier system that can be used is a micro-emulsion carrier system. Such a system can comprise from about 91% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other non-ionics; and from about 7% to about 20% water.

Another exemplary vehicle for topical delivery is liposomes. Liposomes can be used to carry and deliver an agent, e.g., a composition described herein, into a cell. Detailed guidance can be found in, e.g., Yarosh et al. (2001) Lancet 357: 926 and Bouwstra et al. (2002) Adv. Drug Deliv. Rev. 54 Suppl 1:S41.

Topical compositions can also be formulated as a gel or a cosmetic stick, by the addition of a suitable amount of a thickening agent to a cream or lotion formulation.

Topical compositions can also be formulated as makeup products, such as foundations. Foundations are solution or lotion-based with appropriate amounts of thickeners, pigments and fragrance.

Various water-soluble materials can also be present in the formulations. These include humectants, proteins and polypeptides and preservatives. In addition, the topical compositions can include conventional cosmetic adjuvants, such as dyes, opacifiers (e.g., titanium dioxide), pigments and perfumes.

In other embodiments, the topical compositions include a safe and effective amount of a penetration enhancing agent, such as described in U.S. Pat. No. 6,068,834. Other conventional skin care product additives may also be included in the compositions. For example, collagen, hyaluronic acid, elastin, hydrolysates, primrose oil, jojoba oil, epidermal growth factor, soybean saponins, mucopolysaccharides, and mixtures thereof may be used.

In other instances, a topical composition includes one or more sun screening agents. A wide variety of conventional sun screening agents are disclosed in, for example, Cosmetics, Science and Technology 2nd Edition (1972), Vol. 1, Chapter VIII, p. 189. See also U.S. Pat. No. 6,068,834.

An agent may also be added to any of the compositions to improve the skin substantivity of those compositions, particularly to enhance their resistance to being washed off by water or rubbed off. A nonlimiting example is a copolymer of ethylene and acrylic acid, such as disclosed in U.S. Pat. No. 4,663,157.

For systemic administration, a composition described herein may be administered via the oral route or the parenteral route, including subcutaneously, intraperitoneally, intramuscularly, intravenously or other route. For local administration, they can be administered topically, transdermally, transmucosally, intranasally or other route. A cell may be contacted extracellularly or intracellularly with a composition described herein, e.g., by microinjection. More than one route of administration may be used simultaneously, e.g., topical administration in association with oral administration. Examples of parenteral dosage forms include aqueous solutions of the composition in an isotonic saline, 5% glucose or other well-known pharmaceutically acceptable excipient.

The composition may be provided as, e.g., a cosmetic, a medication or a skin care product. The composition can also be formulated into dosage forms for other routes of administration utilizing conventional methods. A composition can be formulated, for example, in dosage forms for oral administration as a powder or granule, or in a capsule, a tablet (each including timed release and sustained release formulations), a drink (e.g., an energy drink), or a gel seal, with optional pharmaceutical carriers suitable for preparing solid compositions, such as vehicles (e.g., starch, glucose, fruit sugar, sucrose, gelatin and the like), lubricants (e.g., magnesium stearate), disintegrators (e.g., starch and crystalline cellulose), and binders (e.g., lactose, mannitol, starch and gum arabic). When the composition is an injection, for example, solvents (e.g., distilled water for injection), stabilizers (e.g., sodium edetate), isotonizing agents (e.g., sodium chloride, glycerin and mannitol), pH-adjusting agents (e.g., hydrochloric acid, citric acid and sodium hydroxide), suspending agents (e.g., methyl cellulose) and the like may be used.

In other instances, the composition is included in a dressing, bandage, transdermic medical device, controlled drug release medical device, or a drug-eluting stent. Particular dressings include hydrocolloid dressings, hydrocellular dressings, alginate dressings, hydrogel dressings, chitosan based dressings, cellulose derivatives dressings and any other type of dressing used for wound protection and repair.

### Methods of Using Stem Cell Compositions

The compositions described herein are useful for improving the condition or aesthetic appearance of skin of a subject. For example, a composition described herein can be used to treat aging skin, wounds, burns (e.g., radiation burns), inflammation, and ulcers. In addition, the compositions described herein can be used to modulate immune responses, to stimulate hematopoiesis, to produce vitamins A, B1, B2, B6, B12, C, or E, or can be used for their anti-neoplastic and antiviral properties, e.g., in a subject. Further uses include to treatment of ulcers and irritable bowel syndrome.

In certain instances, a composition described herein can be applied to an affected area of skin of a subject. Such application may be carried out, for example, by topically applying a cosmetic composition as described herein according to the routine technique for administering such compositions. For example, topical cosmetic compositions can be applied once daily for a period of at around one week, but may include a period of about 2, 4, 8, or 12 weeks. The cosmetic composition can be applied, e.g., to the face, neck, or any area of skin in need thereof, using routine methods. Routine and commonly practiced techniques encompass the application of creams, lotions, gels, masks, sera, ointments, patches, makeup, makeup-removing milks, sunscreen compositions, or the like, to the skin.

The dosage may depend on many factors that are well known to those skilled in the art, for example, the particular form of the composition, the condition being treated, the age, weight, and clinical condition of the recipient subject, and the experience and judgment of a clinician or practitioner administering the composition.

A composition described herein can be used, e.g., to reduce dermatological signs of aging (including, e.g., wrinkles, fine lines, and sagging skin), to increase the number of skin cells, to repair skin cells, to increase signaling for the formation of new skin cells, to increase pliability of skin, to induce or increase collagen formation, to reduce oxidation, to stimulate fibroblast formation and/or connective tissue formation, and/or to increase the production of oils or moisture-enhancing components. A composition or extract described herein can also be used as a demethylating agent or to prevent additional demethylation associated with sun exposure and/or aging. The activity of compositions described herein can be readily measured using assays known in the art, such as visual inspection (e.g., of gloss and/or tenseness of the skin) or measuring for known molecular markers.

A composition described herein can be administered to or used by any subject. Subjects include, but are not limited to, mammals, e.g., humans, other primates, pigs, rodents such as mice and rats, rabbits, guinea pigs, hamsters, cows, horses, cats, dogs, sheep and goats.

In some embodiments, the methods include evaluating the subject for one or more of: dermatological signs of aging (including, e.g., wrinkles, fine lines, and sagging skin), number of skin cells, repair of skin cells, level of a signaling molecule for the formation of new skin cells, pliability of skin, level of collagen formation, level of oxidation, level of fibroblast formation and/or connective tissue formation, and/or level of oils or moisture-enhancing components.

The evaluation can be performed before, during, and/or after the administration of a composition described herein. For example, the evaluation can be performed at least 1 day, 2 days, 4, 7, 14, 21, 30 or more days before and/or after the administration of a composition described herein.

In other instances, an extract or composition described herein can be included in a cell or tissue culture medium. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); salts (e.g., calcium, magnesium and phosphate); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68)). Nonlimiting examples of media that can be supplemented with an extract or composition described herein include Minimal Essential Medium (MEM, Sigma, St. Louis, Mo.); Ham's F10 Medium (Sigma); Dulbecco's Modified Eagles Medium (DMEM, Sigma); RPM 1-1640 Medium (Sigma); HyClone cell culture medium (HyClone, Logan, Utah); and chemically-defined (CD) media, which are formulated for particular cell types, e.g., CD-CHO Medium (Invitrogen, Carlsbad, Calif.).

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1

### Preparation of Aloe vera Stem Cell Composition

Aloe vera shoots are collected from young healthy plants that are disease and pest free. Preferably, Aloe vera shoots are collected from plants with a high acemannan level.

Shoots are cut into pieces of 2-5 cm. The 2-5 cm pieces are washed 20 times by rinsing the cut shoot for 10 minutes each wash in sterile DI water. The pieces are then placed in a 50 mL conical tube containing propidium iodine and washed for 5 minutes with shaking The pieces are then washed with sterile DI water for 10 minutes 20 times with shaking Optionally, an additional wash is performed with a detergent such as tween-20.

The 2- 5 cm pieces are then placed in a conical tube of 1× Penn/Strep and an antifungal agent such as 1× gentamycin in sterile DI water for 1 hour. The pieces are then washed for 1 minute in a solution containing 1.5% v/v Chlorhexidine Gluconate Solution and B.P. Strong cetrimide solution eq. to Cetrimide I.P. 3.0% w/v. The pieces are then washed in sterile DI water for 2 minutes 3 times, after each sterile DI water wash the pieces are dipped in 70% ethanol for 15 seconds.

The 2-5 cm pieces are then washed with a solution of 0.1% mercuric chloride for 5 minutes, followed by 5 washes for 2 minutes each with sterile DI water. The 2-5 cm pieces are then transferred to sterile culture plates and the external leaves are removed.

Next, the pieces are transferred to culture plates containing MS medium (Murashige/Skoog) or another basal medium such as Gamborgs, Vacin/Went, Whites, Schenk/Hildebrandt containing 3% sucrose, 0.2 mg/L BA (6-benzylaminopurine) and 0.2 mg/L IBA (3-indolebutyric acid) , 0.8% agar. Additional additives include Thiamine HCL, Nicotinic acid, Pyridoxine HCL, Glycine, Myo-inositol, and Sucrose (each additive may range at a concentration of 0.1-30000 mg/L).

Aloe vera stem cell cultures are then transferred to a culture environment under a 16 hour photoperiod, with a light intensity of 2000-2500 lux at 25 C. After 10-30 days post Aloe vera stem cell culture initiation, the stem cell cultures are collected for extract preparation.

### Shoot Proliferation

Optionally, the culture is extended to initiate shoot proliferation. For shoot proliferation, stem cell cultures are induced by transferring stem cell cultures in MS medium as previously described with BA (0 -1 mg/L, Kn (Kinetin) (0-1 mg/L), IBA (0.05-0.2 mG/L), Citric acid (0.05-100 mg/L) adenine sulphate (0.1-200 mg/L) and agar (0-0.8%)—(liquid or solid MS medium). After 10-60 days post Aloe vera stem cell culture initiation, stem cells cultures with newly formed shoots are collected for extract preparation.

### Rooting Initiation

Optionally, the culture is further extended to initiate rooting of the microshoots. For microshoot rooting, microshoots are transferred into solid MS medium (0.8% agar containing) that does not contain IBA. 15-30 days post initiation of rooting of microshoots, Aloe vera explants are collected for extract preparation.

After rooting is established, aloe explants are transferred to a garden soil and fertilizer mixture (1:1) for hardening and housed at a humidity of 80% for 5-15 days at 30-35C. 15-30 days post initiation of hardening, Aloe vera explants are collected for extract preparation.

Examples of Macro Salts Used in MS Medium (mg/L)

| | |
|---|---|
| KNO₃ | 1900 |
| NH₄NO₃ | 1650 |
| MgSO₄—7H₂O | 370 |
| CaCl₂—2H₂O | 440 |
| KH₂PO₄ | 170 |

Examples of Micro Salts Used in MS Medium (mg/L)

| | |
|---|---|
| MnSO₄—H₂O | 22.3 |
| ZnSO₄—7H₂O | 8.6 |
| H₃BO₃ | 6.2 |
| KI | 0.83 |
| Na₂MoO₄—2H₂O | 0.25 |
| CuSO₄—5H₂O | 0.025 |
| CoCl₂—6H₂O | 0.025 |
| Na₂Fe-EDTA | 37.24 |

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A cosmetic composition comprising about 25% to about 90% of a succulent plant stem cell extract by weight of the composition.

2. The composition of claim 1 wherein the plant extract is derived from at least one succulent family selected from the group consisting of: Aloaceae, Agavaceae, Cactaceae, Crassulaceae, Aizoaceae, Apocynaceae, Didiereaceae, Euphorbiaceae, Asphodelaceae, and Portulacaceae.

3. The composition of claim 1 wherein the composition further comprises a carrier selected from a group consisting of an emollient, an emulsion, a liposome, and a gel.

4. The composition of claim 1 wherein the composition is administered as a cosmetic, subcutaneously, intraperitoneally, topically, transdermally, transmucosally, intramuscularly, intranasally or intravenously.

5. A cosmetic formulation comprising:
an extract from an Aloe vera callus weighing about 0.5 g to about 1 g; and
an excipient.

6. A method of preparing a cosmetic composition comprising:
growing an Aloe vera callus to a weight of about 0.5 g to about 1 g;
preparing an extract from the callus; and
mixing the extract with an excipient, thereby preparing a cosmetic composition.

7. A cosmetic composition prepared by the method of claim 6.

8. The method of claim 6 wherein the extract to excipient ratio is from about 5% to about 99% by weight.
